(19) United States
(12) Patent Application Publication
(10) Pub. No.: US 2001/0053359 A1
(43) Pub. Date: Dec. 20, 2001

(54) Drug delivery composition for the nasal administration of antiviral agents

(76) Inventors: Peter Watts, Nottingham (GB); Lisbeth Illum, Nottingham (GB)

Correspondence Address: Patrea L Pabst, Holland & Knight LLP, One Atlantic Center Suite 2000, 1201 West Peachtree Street N E, Atlanta, GA, 30309-3400, US

(21) Appl. No.: 09/848,600

(22) Filed: May 3, 2001

Related U.S. Application Data
(63) Continuation of application No. 08/776,470, filed on Mar. 28, 1997, now abandoned.
Application No. 08/776,470 is a 371 of international application No. PCT/GB95/01735, filed on Jul. 24, 1995.

(30) Foreign Application Priority Data
Jul. 26, 1994 (GB) 9414966.3

(51) Int. Cl. A61K 39/395; A61K 9/00

(52) U.S. Cl. 424/130.1; 424/43

(57) ABSTRACT

A drug delivery composition for nasal administration is provided which comprises the antiviral agent ICAM-1 and a bioadhesive material. The bioadhesive material may be a chitosan solution, a liquid formulation comprising a polymeric material or a plurality of bioadhesive microspheres. The polymeric material is preferably gellan gum or alginate. The microspheres may comprise starch, chitosan, hyaluronic acid, or gelatin.

The present invention relates to compositions for nasal administration and, more particularly, to compositions for nasal administration of the antiviral agent known as Intercellular Adhesion Molecule 1 (ICAM-1).

Viral infections affecting the nasal cavity such as influenza and rhinoviral infections can be not only unpleasant disease conditions in normal individuals, but in certain “at risk” groups represent a serious threat to health.

A variety of agents are now available that can be considered as a possible mode of treatment. These include low molecular weight antiviral agents such as Enviroxine, Pirodavir as well as antiviral proteins such as interferon-alpha and sialidase inhibitors (see for example Von Itzstein et al. Nature. 363 418, 1993). More recently it has been shown that rhinoviruses attach to tissue via a specific adhesion process and consequently the binding of virus can be prevented using a cell adhesion molecule such as ICAM-1 or its fragments. (Martin et al., A soluble form of intercellular adhesion molecule-I inhibits rhinovirus infection, Nature 344, 1990, 70-72).

While such antiviral materials can be shown to be effective in vitro using appropriate tests on virus inhibition or binding blockage, it is found that such systems are not effective in vivo for example using the nasal route of administration or have to be given in high and frequent doses that can given rise to toxic effects (Hayden et al. New Eng. J. Med. 314 71 (1986)) or could be disadvantageous on cost grounds. (Al-Nakib, W. et al. Antimicrobial Agents and Chemotherapy 33, 522 (1989)).

Therefore, it would be a significant advantage if it was found possible to increase the effectiveness of antiviral compounds in the nasal cavity. Various nasal delivery systems have previously been described. The nasal administration of starch microspheres has been described by Illum in various patents or patent applications. In U.S. Pat. No. 4,847,091 she described how the low molecular weight drug, sodium cromoglycate, could be complexed to the surface of DEAE-dextran microspheres in order to increase residence time in the nose. PCT/GB88/00836, PCT/GB90/101676 describe how microspheres can be used to increase the systemic uptake of poorly transported molecules such as peptides and proteins.

The use of Chitosan as a bioadhesive material to improve the absorption of polar drugs from the nasal cavity and across other mucosal surfaces has been described by Illum in Topics in Pharmaceutical Science 1991. Editors: Crommellin D. J. A. and Midha, K. K., Stuttgart, Medpharm., 1992. p 71 and in PCT/GB90/00291. Chitosan systems for local application have been described by Partain (U.S. Pat. No. 4,946,870). He described systems that formed substantive films in contact with topical surfaces. The various examples provided by Partain are intended for application as lotions to the skin. Intranasal administration is mentioned in U.S. Pat. No. 4,946,870 but no examples of systems that are not film forming in the nasal cavity are declared. It is also noted that in order to form coherent and substantive films the Partain examples contain high quantities of volatile material such as ethanol. This method would be precluded in the nasal application of such systems.

The use of gellan gum as an in-situ gelling material has been described in AUS 86.63189. with reference to ophthalmic applications The drugs which could be administered by means of the ophthalmic composition according to the invention included antiviral agents such as acyclovir, adenosine, arabinoside, interferon and interferon inducing agents. The pharmaceutical uses of gellan gums and their rheological properties have been described by Deasy et al. Int. J. Pharm. 73 117 (1991) and Kublik and Muller, Eu. J. Pharm. Biopharm 39 192 (1993) and Sanzgiri et al. J. Control. Rel. 26 195 (1993).

We have now found that the effectiveness of the antiviral agent ICAM-1 in the nasal cavity can be greatly increased by administration with a bioadhesive material.

The present invention therefore provides a drug delivery composition for nasal administration comprising ICAM-1 and a bioadhesive material.

We use the term “bioadhesive” to include a material that adheres to the nasal mucosa by chemical or physical binding such as Van der Waals interaction, ionic interaction, hydrogen bonding or by polymer chain entanglement. The adhesion may taken place to the epithelial (cellular) surface or to the mucus overlying that surface.

Rhinoviruses belong to the picornavirus family and cause about 50% of common colds. Most rhinoviruses share a common receptor on human cells. The glycoprotein inter cellular adhesion molecule-1 (ICAM-1) has recently been identified as the cellular receptor for the subgroup of rhinoviruses known as the major group. ICAM-1 is expressed on cells of multiple lineages at sites of inflammation. ICAM-1 is glycosylated protein with a molecular weight of 85-90 kD, the protein portion having a molecular weight of 45-50 kD.

In a first embodiment of the invention, the bioadhesive material is chitosan, preferably as a solution. The chitosan solution may be made in water or any suitable pharmaceutically acceptable buffer system. Phosphate or lactate buffers are especially preferred.

The concentration of chitosan in the solution is preferably in the range 0.01 to 50% w/v, more preferably 0.1 to 30%, more preferably 0.1% to 15% and most preferably 0.2% to 2.0%.

Chitosan is deacetylated chitin, or poly-N-acetyl-D-glucosamine. It is available from Protan Laboratories Inc, Redmond, Wash. 98052 and, depending on the grade selected, can be soluble in water up to pH 6.0. A 1% solution of non-water soluble chitosan (Sea Cure) may be made by making a slurry (eg. 2 g/100 ml) in water and adding an equal volume of organic acid (eg 100 ml of 2% acetic acid) and stirring vigorously for one hour. Water-soluble chitosan (see Cure⁺) may dissolve without organic or inorganic acids being present.

Chitosan has previously been used to precipitate proteinaceous material, to make surgical sutures and as an immunostimulant. It has also been employed previously in oral drug formulations in order to improve the dissolution of poorly soluble drugs (Sawayanagi et al, Chem. Pharm. Bull., 31, 2062-2068 (1983)) or for the sustained release of drugs (Nagai et al, Proc. Jt. U.S.-Jpn. Semin. Adv. Chitin, Chitosan, Relat. Enzymes, 21-39. Zikakis J. P. (ed), Academic Press. Orlando (1984)) by a process of slow erosion from a hydrated compressed matrix.

The chitosan formulation may also contain a preservative such as benzalkonium chloride. The ICAM-1 is preferably mixed with the chitosan solution in concentrations in the range of 0.01% to 20% w/v, preferably 0.1% to 10% w/v, and more preferably 0.2% to 5% w/v. The Chitosan formulation can be administered using a conventional nasal spray device familiar to those skilled in the art.

In a second embodiment of the invention, the bioadhesive material is a plurality of microspheres.

The microsphere can be prepared from a suitable material such as starch, starch derivatives, amylodextrin, amylopectin and cross-linked variants thereof, gelatin, albumin, alginate, gellan, hyaluronic acid, chitosan, dextran and dextran derivatives. The microspheres may also comprise ion-exchange materials. By the term “derivatives” we particularly mean esters and ethers of the parent compound that can be unfunctionalised or functionalised to contain, for example, ionic groupings.

Suitable starch derivatives include hydroxyethyl starch, hydroxypropyl starch, carboxymethyl starch, cationic starch, acetylated starch, phosphorylated starch, succinate derivatives or starch and grafted starches. Such starch derivatives are well known and described in the art (for example Modified Starches: Properties and Uses, O. B. Wurzburg, CRC Press Boca Raton (1986)).

Suitable dextran derivatives include diethylaminoethyl-dextran (DEAE-dextran), dextran sulphate, dextran methyl-benzylamide sulphonates, dextran methyl-benzylamide carboxylates, carboxymethyl dextran, diphosphonate dextran, dextran hydrazide, palmitoyldextran and dextran phosphate. These microspheres can be prepared by emulsification procedures or by spray drying. Both are established procedures in pharmaceutical formulation and are familiar to those skilled in the art. The microspheres are preferably of a size from 1 to 200 micron, more preferably 10-100 microns, and most preferably 40-60 μm. Substantially uniform, solid microspheres are preferred

The drug-microsphere formulations are prepared as a freeze-dried or spray dried powder system or a physical mixture. The microspheres can be administered by a nasal insufflator or a device that would be normally used for deposition of powders into the lungs but suitably modified for nasal administration. Examples include the Ventolin inhaler (from Glaxo) and the Dura Dry Powder Device (U.S. Pat. No. 5,327,883). Bespak device (WO935950).

The bioadhesive microspheres have the property of swelling in water. This swelling nature leads to a preferential binding to the mucosal surface of the nose thereby leading to improved retention. The degree of swelling should be such that the particle increases its diameter (as measured by a suitable technique such as the light microscope, laser diffractomer) when immersed in water by a factor of at least 1.2 times. The preferable increase in diameter is 1.5 times or greater.

The formulation can either be prepared by freeze drying from a suspension of the microspheres in drug solution or by mechanically mixing the freeze dried, spray dried or dried microspheres with the drug in a powder form. The drug can be sorbed into or onto the microspheres after their preparation. Alternatively the drug can also be incorporated into the microspheres during their production. The technique of spray drying or an emulsification technique or other techniques known to the person skilled in the art can be used to produce microspheres of the desired size that contain the drug. The conditions or preparation are selected by the person skilled in the art to provide particles that have the necessary integrity and also to maintain the biological activity of the drug. Preparation of these microsphere systems is well described in the pharmaceutical literature (see for example Davis et al, (Eds), “Microspheres and Drug Therapy”, Elsevier Biomedical Press, 1984).

Emulsion and phase separation methods are both suitable. For example, albumin microspheres may be made using the water-in-oil emulsification method where a dispersion of albumin is produced in a suitable oil by homogenization techniques or stirring techniques, with the addition if necessary of small amounts of an appropriate surface active agent.

Emulsification techniques are also used to produce starch microspheres as described in GB 1 518 121 and EP 223 303 as well as for the preparation of microspheres of gelatin. Proteinaceous microspheres may also be prepared by coacervation methods such as simple or complex coacervation or by phase separation techniques using an appropriate solvent or electrolyte solution. Full details of the methods of preparing these systems can be obtained from standard text books (see for example Florence and Attwood, Physicochemical Principles of Pharmacy 2nd Ed., MacMilan Press, 1988, Chapter 8).

The microspheres can be hardened by well known cross-linking procedures such as heat treatment or by chemical cross-linking agents. Suitable agents include dialdehydes, including glyoxal, malondialdehyde, succinicaldehyde, adipaldehyde, glutaraldehyde and phthalaldehyde, diketones such as butadione, epichlorohydrin, polyphosphate and borate. Dialdehydes are used to cross-link proteins such as albumin by interaction with amino groups and diketones form schiff bases with amino groups. Epichlorohydrin activates compounds with nucleophiles such as amino or hydroxyl to an epoxide derivative.

For example, microspheres were made as follows:

Starch Microspheres

15 ml of 5% starch solution (pH=7) was kept at a constant temperature of 70° C. and stirred (500 rpm) while a 30% solution of PEG was added (about 7 ml) until phase separation had occurred. The system was then stirred for a further 15 min, before it was cooled on ice during constant stirring. The microspheres were then isolated by filtration and freeze dried. With a stirring speed of 500 rpm, particles with a mean size of 33 μm±10 μm were produced.

Gelatine Microspheres

30 ml of 10% bovine gelatin (pH=8.5) was kept at a constant temperature of 50° C. and stirred (500 rpm) while a 30% solution of PEG was added (about 20 ml) until the coacervation region was reached. To control this step, a nephelometer can be used. The mixture was cooled on ice during constant stirring. The microspheres were isolated by filtration and freeze dried.

With a stirring speed of 500 rpm, particles with a mean size of 60 μm±10 μm were produced.

The content of ICAM-1 in the microsphere formulation is preferably in the range 0.1% to 50% w/w, more preferably 0.5% to 25% and most preferably 1% to 20% w/w.

In a third embodiment of the invention the bioadhesive material may be a liquid formulation comprising a polymeric material.

The polymeric material should provide a viscous solution to aid retention in the nasal cavity. Preferably, the polymeric material will gel when in contact with the nasal mucosa either due to the rise in temperature, the presence of specific cations or change in pH.

Suitable polymeric materials include gellan gum, welan, rhamsan, alginate, carboxymethylcellulose, sodium alginate, xanthan, agar, guar derivatives such as carboxymethyl guar gum, carageenan, dextran sulphate, keratan, dermatan, pectin. Polysaccharides and derivatives are particularly suitable (“Polysaccharides and derviatives” edited by R C Whistler and J N BeMiller (3rd Ed.) Academic Press, San Diego 1993).

A preferred material is gellan gum, which is the deacetylated form of the extracellular polysaccharide from Pseudomonas elodae. Native/high-acyl gellan is composed of a linear sequence of tetra-saccharide repeating units containing D-glucuronopyranosyl, D-glucopyranosyl and L-rhamnopyranosyl units and acyl groups.

Another preferred material is alginate. Alginate is composed of two building blocks of monomeric units namely β-D-mannuronopyranosyl and α-guluronopyranosyl units. The ratio of D-mannuronic acid and L-guluronic acid components and their sequence predetermines the properties observed for alginates extracted from different seaweed sources.

Welan is produced by an Alcaligenes species. Welan has the same basic repeating unit as gellan but with a single glycosyl sidechain substituent. The side unit can be either an α-L-rhamnopyranosyl or an α-L-mannopyranosyl unit linked (1→3) to the 4-0-substituted β-D-glucopyranosyl unit in the backbone.

Rhamsan is produced by an Alcaligenes species. Rhamsan has the same repeating backbone unit as that of gellan but with a disaccharide side chain on O-6 of the 3-O-substituted β-D-glucopyranosyl unit. The side chain is a β-D-glucopyranosyl-(1-6)-α-D-glucopyranosyl unit.

Xanthan is produced by a number of Xanthomonas strains. The polymer backbone, made up of (1→4)-linked β-D-glucopyranosyl units is identical to that of cellulose. To alternate D-glucosyl units at the 0-3 position, a trisaccharide side chain containing a D-glucoronosyl unit between two D-mannosyl units is attached. The terminal β-D-mannopyranosyl unit is glycosidically linked to the 0-4 position of the β-D-glucopyranosyluronic acid unit, which in turn is glycosidically linked to the 0-2 position of an α-D-mannopyranosyl unit.

Carrageenan is a group of linear galactan polysaccharides extracted from red seaweeds of the Gigartinaceae, Hypneaceae, Solieriaceae, Phyllophoraceae and Furcellariaceae families that have an oster sulfate content of 15-40% and contain alternatively (1→3)-α-D- and (1→4)-α-D-glycosidic linkages.

Agar is a hydrophilic colloid extracted from certain marine algae of the class Rhodophyceae where it occurs as a structural carbohydrate in the cell walls (see also Kang and Pettitt: Xanthan, Gellan, Welan and Rhamsan in Industrial gums by Whistler and BeMiller (Eds), Academic Press Inc. London, 1993).

Mixtures of gellan with other polymers such as alginate can be used, gelling of the mixture being caused by the gellan gum. Other combinations of gums can also be used, particularly where the combination gives a synergistic effect, for example in terms of gelation 30 properties. An example is xanthan—locust bean gum combinations.

The advantage of gellan over other materials is that it can be administered as a fluid system but in the nasal cavity the system will gel, thereby providing a bioadhesive effect and holding the drug at the absorptive surface for an extended period of time.

The grade of gellan gum can be Gelrite or Kelcogel from Kelco Int, Ltd. or other similar grades from other manufacturers. The gellan can be prepared at a concentration of 0.1 w/v to 15% but a preferred range of concentrations is 0.2% to 1%.

For some of the polymer materials monovalent or divalent cations must be present in the composition for gelling to occur. Such polymer materials include gellan gum, welan, rhamsan and alginate.

Suitable cations include sodium, potassium, magnesium and calcium. The ionic concentration is chosen according to the degree of gelling required, and allowing for the effect that the ionised drug present may have on gelling. At a 0.2% gum concentration, the divalent ions, calcium and magnesium give maximum gel hardness and modulus at molar concentrations approximately one fortieth (1/40) of those required with the monovalent ions, sodium and potassium. A finite concentration of each cation is required to induce gelation. For the nasal formulations of the invention the ionic strength is kept sufficiently low to obtain a low viscosity formulation but sufficiently high to ensure gelation once administration into the nasal cavity where gelation will take place due to the presence of cations int he nasal liquid. The ionic strength for a 0.5% gellan gum can be in the range of 0.1 mM-50 mM for monovalent cations with the preferred range being 1 mM-5 mM and 0.1 mM-5 mM for divalent cations with the preferred range being 0.15 mM-1 mM. For higher concentrations of gellan gum the ionic strengths should be lowered accordingly.

In a liquid formulation, the polymeric material will typically be provided in a concentration of from 0.01% to 20%, preferably 0.05-10%, more preferably 0.1%-5%. The compositions of the invention can also contain any other pharmacologically acceptable, non-toxic ingredients such as preservatives, antioxidants, flavourings, etc. Benzalkonium chloride may be used as a preservative. The ICAM-1 is used in concentrations in the formulation in the range of 0.01% to 20% w/v, more preferably 0.1% to 10% w/v and most preferably 0.2% to 5% w/v.

The liquid formulations are administered using well-known nasal spray devices. If the formulations are freeze-dried, they can be administered using a nasal insufflator, as for the microsphere preparations.

It has been found that by administering ICAM-1 to the nasal cavity with the bioadhesive material according to the invention, the effectiveness of the ICAM-1 in the nasal cavity is greatly increased. It is thought that this is due to the delay in mucociliary clearance of the ICAM-1 from the nasal cavity which is caused by the bioadhesive material, and also the controlled release of the ICAM-1 from the bioadhesive material.

Preferred embodiments of the invention will now be illustrated in more detail by way of the following examples.

### EXAMPLE 1

Into a 20 ml volumetric flask weighed 100 mg of chitosan glutamate (Sea Cure+210). 5 ml of water was added to the chitosan which was left to stir overnight.

Into a beaker was weighed 1.36 g of potassium diyhydrogen phosphate and 2.80 g of sodium chloride. The salts were dissolved in 80 ml of water, the solution adjusted to pH 5.7 using 2N NaOH solution and then made to 100 ml with water. When the chitosan had dissolved, 5 ml of the phosphate buffer solution was added.

A formulation containing 10 mg/ml ICAM and 5 ml/ml chitosan was prepared by diluting the solution of chitosan in phosphate buffer 1:1 with 20 mg/ml ICAM solution.

### EXAMPLE 2

Into a 3 ml glass vial was weighed 10 mg of gellan gum (Keleogel, Kelco Inc). To the glass vial was added 1.8 ml of 11 mg/ml ICAM solution, 0.05 ml of 4 mg/ml benzalkonium chloride solution and 0.15 ml of water.

A small magnetic stirrer bar was added to the vial, and the contents stirred at room temperature for 24 hours to disperse the gellan gum.

### EXAMPLE 3

Into a 250 ml conical flask were weighed 500 mg of “Eldexomer” starch microspheres obtained from Perstorp, Sweden.

To the conical flask containing the microspheres was added 31 ml of water and 1.6 ml of 12.5 mg/ml ICAM solution.

The flask contents were gently mixed and then left to stand for 30 minutes.

The contents of the conical flask were frozen by immersing the flask into liquid nitrogen. The flask was swirled during freezing to obtain a homogeneous mixture.

The flask was transferred to a freeze drier and the contents lyophilised for 24 hours. The resulting product was a free flowing powder containing 1 mg of ICAM/21 mg of formulation.

### EXAMPLE 4

Into a 10 ml volumetric flask was weighed 1.0 g of gelatin which was dissolved in 5 ml of water by warming to 35-40° C.

To the gelatin solution was added 1.8 ml of 22 mg/ml ICAM solution. The flask contents were made to volume with water.

Into a beaker was measured 90 mg of 1% of 1% w/v Span 80 in soya oil. The beaker contents were warmed to 35-40° C. on a hot-plate.

The warmed Span/soya oil mixture was removed from the hot-plate and was stirred at 1000 rpm using an overhead stirrer.

The 10 ml of ICAM/gelatin mixture was added to the stirring oil and stirred at 1000 rpm for 2 minutes.

While stirring continued, the beaker containing the emulsion was cooled to 15° C. by surrounding in ice. Dropwise, 100 ml of acetone was added to the cooled, stirred emulsion.

The gelatin-ICAM microspheres were recovered by centrifugation, washed with acetone and left to dry at room temperature.

The result was a free-flowing powder containing 1 mg of ICAM/26 mg of formulation. The mean diameter of the microspheres was measured, using laser diffraction, to be 50 μm.

### EXAMPLE 5

500 mg of medium viscosity chitosan glutamate was dissolved in 50 ml of water. 26 mg of ICAM was dissolved in the chitosan solution. The chitosan/ICAM solution was spray-dried using a Lab-Plant SD-04 spray-dryer (Lab-Plant, Huddersfield, UK). The drying temperature was set at 100° C. Microspheres of approximately 5 μm diameter was formed.

### EXAMPLE 6

1000 mg of gelatin was dissolved in 50 ml of water at 40° C. 52 mg of ICAM was dissolved in the gelatin solution. The chitosan/ICAM solution was spray-dried using a Lab-Plant SD-04 spray-dryer (Lab-Plant. Huddersfield, UK). The drying temperature was set at 100° C. Microspheres of approximate 5 μm size were formed.

### EXAMPLE 7

An aqueous solution was prepared containing 2.1 mg/ml ICAM, 1.4 mg/ml Mannitol and 0.7 mg/ml PBS. The solution was spray-dried at 100° C. to form microparticles of approximately 5 μm size. 10 mg of spray dried ICAM and 100 mg of Eldexomer starch microspheres were weighed into a bottle and placed on to a roller mixer for 30 minutes. The resulting formulation consisted of starch microspheres coated with particles of spray-dried ICAM.

1. A drug delivery composition for nasal administration comprising ICAM-1 and a bioadhesive material.

2. A drug delivery composition according to claim 1 wherein the bioadhesive material is a chitosan solution.

3. A drug delivery composition according to claim 2 wherein the chitosan is in the solution in a concentration in the range of 0.2-2.0% w/v.

4. A drug delivery composition according to claim 2 or 3 wherein the ICAM-1 is present in the chitosan solution in a concentration in the range 0.2 to 5% w/v.

5. A drug delivery composition according to claim 1 wherein the bioadhesive material is a plurality of microspheres.

6. A drug delivery composition according to claim 5 wherein the microspheres are made from starch, chitosan, gelatin, hyaluronic acid, alginate or gellan.

7. A drug delivery composition according to claim 5 or 6 wherein the ICAM-1 is present in an amount of 1% to 20% w/w of the microspheres.

8. A drug delivery composition according to claim 1 wherein the bioadhesive material is a liquid formulation comprising a polymeric material.

9. A drug delivery composition according to claim 8 wherein the polymeric material is gellan gum, alginate, welan, xanthan or rhamsan.

10. A drug delivery composition according to claim 8 or 9 wherein the polymeric material is provided in a concentration of 0.1% to 5% w/v.

11. A drug delivery composition according to any one of claims 8 to 10 wherein the ICAM-1 is present in the formulation in an amount of 0.2% to 5% w/v.

12. A method of delivering ICAM-1 to the nasal cavity to increase its effectiveness therein comprising administering the ICAM-1 in a drug delivery composition additionally comprising a bioadhesive material.
